(19) United States
(12) Patent Application Publication
(10) Pub. No.: US 2021/0386809 A1
(43) Pub. Date: Dec. 16, 2021

(54) EXTRACTION

(71) Applicant: PHYTONEXT B.V., Wageningen (NL)

(72) Inventors: Mazin NICOLA, Worthing (GB); Eral OSMANOGLOU, Wageningen (NL)

(21) Appl. No.: 16/967,944

(22) PCT Filed: Feb. 12, 2019
(86) PCT No.: PCT/EP2019/053388

§ 371 Date: Aug. 6, 2020

(30) Foreign Application Priority Data
Feb. 16, 2018 (GB) 1802596.5

(51) Int. Cl. A61K 36/185; B01D 11/02

(52) U.S. Cl. CPC A61K 36/185; B01D 11/0288; B01D 11/0292; A61K 2236/31; B01D 11/028; A61K 2236/35; B01D 11/0219

(57) ABSTRACT

A method of extracting at least one cannabinoid from a biomass comprises the following steps (i) contacting the biomass with a solvent formulation which comprises a C₁₋₄ fluorinated hydrocarbon or a C₁₋₄ hydrofluorocarbon ether, thereby to charge the solvent formulation with an extract from the biomass; and (ii) separating charged solvent formulation from the biomass.

This invention relates to extraction, for example of botanical drug substances. Particularly, although not exclusively, the invention relates to preparation of extracts, for example of botanical drug substances (BDS), which comprise bio-cannabinoids.

The cannabis plant has been used for therapeutic as well as recreational purposes for a very long time. The easing of some of the prohibition laws regarding the cultivation and use of cannabis material in recent years has resulted in an ever increasing global demand for cannabis plant products. This includes dried plant biomass, whole extract and single pure cannabinoids or combinations of these. This has led to a marked increase in R&D activity and, consequently, to improved knowledge of the chemistry and medicinal properties of the cannabis plant.

Modern uses fall into the following categories:
Recreational: Plant material, or an extract, with high tetrahydocannabinol (THC) content is used.
Prescription Medicines: These are mixtures comprising one or more purified cannabinoids including THC, CBD and others. They are regulated by the FDA (and other country specific regulatory bodies) through the “New Drug Application” (NDA) process. This is a long and tortuous process that starts with a formal application to the regulatory body to request approval for marketing a new drug. An NDA has to include all animal and human data and analyses of such data, as well as information relating to how the drug behaves in the body and how it is manufactured, its purity and efficacy specification and its impurity profiles.
Botanical Drug Substance (BDS): Botanical Drug Development Guidance for Industry published in December 2016 by U.S. Department of Health and Human Services Food and Drug Administration “Centre for Drug Evaluation and Research” (CDER) describes a BDS as: “A botanical product intended for use in diagnosing, curing, mitigating, or treating disease. It is derived from one or more plants, algae, or microscopic fungi. It is prepared from botanical raw materials by one or more of the following processes: grinding, decoction, expression, aqueous extraction, ethanolic extraction or other similar processes.” The defined criteria excludes, among other things: “Highly purified substances, either derived from a naturally occurring source or chemically modified”. BDSs can often be marketed under an over-the-counter (OTC) drug monograph and may be available as (but not limited to) a solution (e.g. tea), powder, tablet, capsule, elixir, topical application or injection.

Accordingly, a BDS obtained from the cannabis plant does not have to comply with strictly defined specifications of purity as one single compound such as tetrahydocannabinol (THC) or cannabidiol (CBD). A cannabis based BDS may be rich in a single compound and/or may include minor impurities, such as non-cannabinoid impurities. Alternatively, a cannabis based BDS may comprise a mixture of two or more compounds that have synergistic pharmaceutical activity including, but not limited to, THC, CBD, tetrahydrocannabivarin (THCV), Cannabidivarin (CBDV), cannabichromene (CBC) cannabigerol, cannabicyclol, cannabielsol. Furthermore, a cannabis-based BDS may include non-cannabinoid compounds such as flavour and aroma terpenes and terpenoids. For example, a BDS may comprise a mixture of equal amounts of THC and CBD or ratios of THC:CBD of any value between 95%:5% and 5%:95%. Or, the BDS may contain 90%-99.5% total cannabinoids comprising one or more of the following: THC, THCV and CBDV and others, where the total cannabinoids content of the BDS may be (for example) between 50%:50% and 99.5%:0.5%.

Published methods for preparation of cannabinoids from the cannabis plant are multi step consisting of solvent extraction followed by isolation of crude intermediates followed by one or more purification steps. The extraction step may be carried out using an organic solvent such as hexane, ethyl acetate, methanol or ethanol. The solvent is then removed by vacuum aided evaporation at elevated temperatures and a crude intermediate extract is isolated. In general terms, in known extraction processes, a mass of material is subjected to solvent extraction in a first step to produce a first or primary extract from which a number of compounds are isolated as a mixture. Subsequently, in a following second step, the mixture of compounds isolated from the first extract is further treated, for example extracted, to produce a second extract from which is isolated a mixture containing predominantly the desired compounds. Subsequently, in a following third step, the mixture of compounds isolated from the second extract is further treated, for example using chromatography to remove undesired compounds and to produce a mixture containing the desired compounds in a solution with a chromatography mobile phase. The desired compounds are then isolated from the mobile phase by, for example, a distillation method. However, such multiple step processes are time consuming and expensive. Furthermore, subjecting some extracts to further processes is undesirable since it may result in contamination, loss or damage of the desired materials.

It is an object of the present invention to address the above described problems.

It is an object of the invention to provide a simple and efficient process for preparing BDS from cannabis-containing plant material.

According to a first aspect of the invention, there is provided a method of extracting at least one cannabinoid from a biomass, the method comprising the following steps:

(i) contacting the biomass with a solvent formulation which comprises a C₁₋₄ fluorinated hydrocarbon or a C₁₋₄ hydrofluorocarbon ether, thereby to charge the solvent formulation with an extract from the biomass; and

(ii) separating charged solvent formulation from the biomass.

The method has been found to be particularly efficient at extracting high purity cannabinoids from the biomass which thereby may not require extensive downstream purification prior to use as a BDS.

In step (i), contact (e.g. initial contact) of biomass with solvent formulation may take place when the biomass is at a temperature of less than ambient temperature, for example less than 10° C., suitably less than 5° C., preferably less than 0° C., more preferably less than −5° C., especially less than −8° C. Thus, it is preferred that the biomass is cooled to a temperature of less than ambient temperature prior to contact (e.g. initial contact) with said solvent formulation. Such cooling may be achieved by placing the biomass in a refrigerator or freezer. The temperature of said biomass on contact (e.g. initial contact) with said solvent formulation may be at least −20° C., preferably at least −14° C.

The solvent formulation which contacts (e.g. initially contacts) the biomass may be at a temperature of less than ambient temperature, for example less than 15° C., suitably less than 10° C., preferably less than 5° C., more preferably less than 0° C., especially less than −2° C. Said temperature of said solvent formulation may be greater than −20° C., suitably greater than −15° C., preferably greater than −12° C.

Said solvent formulation may be maintained at a temperature as described, especially a temperature of less than 5° C., less than 0° C. or less than −2° C., for a period of at least 10 minutes, preferably at least 30 minutes, more preferably at least 1 hour, especially for at least 1.5 hours. Said solvent formulation may be passed through the biomass multiple times. For example, it may be circulated and/or re-circulated through the biomass, suitably whilst maintaining the temperature of the solvent formulation at a temperature of less than 5° C., preferably less than 0° C., more preferably less than −2° C. The low temperature described is found to be advantageous for extracting desired cannabinoids from the biomass, at high purity.

In the method, said biomass may be arranged in a receptacle between an inlet and outlet of the receptacle. In the method, solvent formulation may pass into the receptacle via said inlet, through the biomass and out of the receptacle via said outlet.

Said biomass arranged in said receptacle may be in any suitable form. The form is suitably selected to optimise extraction of components therefrom. Thus, biomass may be processed to adjust its form for use in the process. For example, solid material may be comminuted and such comminuted material may be arranged in the receptacle. In preferred embodiments, the mass of material arranged in the receptacle is in a finely divided form.

The method of the first aspect may involve selecting a biomass which includes components to be extracted and packing the biomass into free space in said receptacle so that said biomass extends over a length of at least 5 cm, preferably at least 20 cm, more preferably at least 40 cm in said receptacle, preferably in a column. Said biomass preferably extends to an upper region of the receptacle and a lower region thereof and, suitably, only the biomass is present in the receptacle between said upper and lower regions. Only after said mass of material has been packed into said receptacle so that it extends over said length described is a solvent formulation passed into said receptacle.

Said biomass may be packed into said receptacle at a density of at least 0.25 g/cm³, preferably at least 0.30 g/cm³, more preferably at least 0.35 g/cm³, especially at least 0.40 g/cm³. The density of the biomass may be achieved by use of a ram (or the like) to compress the biomass. The biomass is suitably substantially immovable when in position. The biomass is suitably substantially static during the flow of said solvent formulation therethrough.

Said solvent formulation may be passed through the biomass at a rate of at least 0.02 ml/minute per gram of said biomass in the receptacle. Said rate may be less than 1 ml/minute per gram. The flow rate may be at least 0.5 BV/hour where “By” refers to the bed volume. Thus, the unit refers to the volume of said solvent formulation passing through the biomass per hour divided by the volume taken up by the biomass. The flow rate is preferably 10 BV/hour or less.

As described, said solvent formulation comprises a C₁₋₄ fluorinated hydrocarbon or a C₁₋₄ hydrofluorocarbon ether.

A said hydrofluorocarbon ether preferably comprises one or more carbon, fluorine, hydrogen and oxygen atoms only. It may include up to 10, preferably up to 8, more preferably, up to 6, fluorine atoms. It preferably includes at least 2, more preferably at least 3 fluorine atoms. It is preferably aliphatic and/or saturated. An example of a hydrofluorocarbon ether is 1,1,1,2,2-pentafluorethyl methyl ether.

Said C₁₋₄ fluorinated hydrocarbon is preferably non-chlorinated. Preferably, it comprises one or more carbon atoms, one or more fluorine atoms together with one or more other atoms selected from hydrogen atoms and iodine atoms. More preferably, it comprises one or more carbon, fluorine and hydrogen atoms only. Preferably, said fluorinated hydrocarbon is a C₁₋₃, more preferably a C₂₋₃, fluorinated hydrocarbon. Especially preferred is a C₂ fluorinated hydrocarbon.

Said fluorinated hydrocarbon may include 10 or fewer, suitably 8 or fewer, preferably 7 or fewer, more preferably 5 or fewer, especially 4 or fewer, fluorine atoms. Preferably, said fluorinated hydrocarbon includes at least 2, more preferably at least 3, fluorine atoms.

Said fluorinated hydrocarbon is preferably aliphatic. It is preferably saturated.

Said fluorinated hydrocarbon may have a boiling point at atmospheric pressure of less than 20° C., preferably less than 10° C., more preferably less than 0° C., especially less than −10° C. The boiling point may be greater than −90° C., preferably greater than −70° C., more preferably greater than −50° C., especially greater than −40° C.

Said solvent formulation may comprise a solvent selected from: iodotrifluoromethane, CF₃H (HFC-23, trifluoromethane), CH₃F (HFC-41, fluoromethane), CH₂F₂ (HFC-32, difluoromethane), CF₃CF₂H (HFC-125, pentafluoroethane), CF₃CH₃ (HFC-143 A, 1,1,1-trifluoroethane), HCF₂CH₃ (HFC-152 A, 1,1-difluoroethane), CF₃CHFCF₃ (HFC-227 EA, 1,1,1,2,3,3,3-heptafluoropropane), CF₃CF₂CF₂H (HFC-227 CA, 1,1,1,2,2,3,3-heptafluoropropane), CF₃CH₂CF₃ (HFC-236 FA, 1,1,1,3,3,3-hexafluoropropane), CF₃CF₂CH₃ (HFC-245 CB, 1,1,1,2,2-pentafluoropropane), CF₃CF₂CH₂F (HFC-236 CB, 1,1,1,2,2,3-hexafluoropropane), HCF₂CF₂CF₂H (HFC-236 CA, 1,1,2,2,3,3-hexafluoropropane), CF₃CHFCF₂H (HFC-236 EA, 1,1,1,2,3,3-hexafluoropropane), and CH₂FCF₃ (HFC-134A, 1,1,1,2-tetrafluoroethane).

Preferably, said solvent formulation comprises a solvent selected from: iodotrifluoromethane, 1,1,1,2,3,3,3-heptafluoropropane (HFC-227 EA), 1,1,1,2,2,3,3-heptafluoropropane (HFC-227CA) and 1,1,1,2-tetrafluoroethane (HFC-134a).

More preferably, said solvent formulation comprises a solvent selected from: 1,1,1,2,3,3,3-heptafluoropropane (R-227EA) and 1,1,1,2-tetrafluoroethane, with 1,1,1,2-tetrafluoroethane being especially preferred.

Said C₁₋₄ fluorinated hydrocarbon or C₁₋₄ hydrofluorocarbon ether preferably has a purity of at least 98% w/w.

Said solvent formulation is preferably in a liquid state when contacted with said biomass in step (i) of the method. Said solvent formulation is preferably in a sub-critical state when contacted with said biomass in the method.

Said solvent formulation preferably includes a major amount of C₁₋₄ fluorinated hydrocarbon or C₁₋₄ hydrofluorocarbon ether. Said solvent formulation suitably includes at least 70 wt %, preferably at least 80 wt %, more preferably at least 90 wt %, especially at least 92 wt % of a said C₁₋₄ fluorinated hydrocarbon or C₁₋₄ hydrofluorocarbon ether. Said solvent formulation preferably includes a single type of C₁₋₄ fluorinated hydrocarbon or C₁₋₄ hydrofluorocarbon ether. Said solvent formulation preferably includes a single type of C₁₋₄ fluorinated hydrocarbon and no C₁₋₄ hydrofluorocarbon ether. Said solvent formulation preferably includes a major amount (e.g. at least 70 wt %, preferably at least 80 wt %, more preferably at least 90 wt %, especially at least 92 wt % of a C₁₋₄ fluorinated hydrocarbon, especially HFC 134a.

In some embodiments, said solvent formulation may include at least 95 wt %, preferably at least 97 wt %, more preferably at least 99 wt % of a C₁₋₄ fluorinated hydrocarbon, especially HFC 134a. In such embodiments, said solvent formulation may consist essentially of a single C₁₋₄ fluorinated hydrocarbon, especially HFC 134a.

Where the solvent formulation does not consist essentially of a single solvent, the solvent formulation may include a modifier to adjust the properties of the solvent formulation.

Said modifier may comprise any material which is capable of modifying the properties of the solvent formulation thereby to affect extracts obtained from the biomass. Said selected modifier may affect the pH of the solvent formulation. Preferably, said selected modifier comprises a co-solvent. A said co-solvent may be an additional C₁₋₄ fluorinated hydrocarbon or C₁₋₄ hydrofluorocarbon ether. Preferably, said solvent formulation includes a modifier selected from: a C₂₋₆ hydrocarbon such as an alkane or cycloalkane with alkanes such as ethane, n-propane, i-propane, n-butane and i-butane being especially preferred; and hydrocarbon ethers, particularly dialkylethers such as dimethylether, methylethylether and diethyl ether. In other embodiments, said modifier may be polar, for example having a dielectric constant, at 20° C., of greater than 5. Such modifier may be selected from: amides, especially N,N′-dialkylamides and alkylamides, with dimethylformamide and formamide being preferred; sulphoxides, especially dialkyl sulphoxides, with dimethylsulphoxide being preferred; alcohols, especially aliphatic alcohols for example alkanols, with methanol, ethanol, 1-propanol and 2-propanol being preferred; ketones, especially aliphatic ketones, for example dialkyl ketones, with acetone being especially preferred; organic acids, especially carboxylic acids with formic acid and acetic acid being preferred; carboxylic acid derivatives, for example anhydrides, with acetic anhydride being preferred; cyanide derivatives, for example hydrogen cyanide and alkyl cyanides, with methyl cyanide and liquefied anhydrous hydrogen cyanide being preferred; ammonia; sulphur containing molecules including sulphur dioxide, hydrogen sulphide and carbon disulphide; inorganic acids for example hydrogen halides with liquefied anhydrous hydrogen fluoride, chloride, bromide and iodide being preferred; nitro derivatives, for example nitroalkanes and nitroaryl compounds, with nitromethane and nitrobenzene being especially preferred.

A preferred modifier may have a boiling point of at least −30° C., for example −26° C.; and preferably less than 10° C., or less than 2° C. A preferred modifier may have a melting point of greater than −160° C., for example greater than −150° C.; and preferably less than −100° C. or less than −120° C. A preferred modifier includes carbon and hydrogen atoms and, optionally, an oxygen atom. A preferred modifier is saturated. A preferred modifier is selected from an alkane and an ether. A preferred modifier has a molecular weight of at least 30, preferably at least 40, more preferably at least 44; and a molecular weight of less than 100, preferably less than 80, more preferably less than 65.

Said solvent formulation may include up to 20 wt %, preferably up to 10 wt %, more preferably up to 8 wt % of modifier (preferably a single type of modifier). In some embodiments, said solvent formulation may include no modifier.

Said solvent formulation may include only a single type of modifier. Said solvent formulation may consists essentially of a single solvent selected from a C₁₋₄ fluorinated hydrocarbon (said solvent preferably being HFC 134a) and C₁₋₄ hydrofluorocarbon ether and a single modifier (said modifier preferably being an alkane or ether, with butane and dimethylether being especially preferred).

The conditions referred to above under which said biomass is contacted with said solvent formulation are referred to as a or said “first set of conditions”.

After contact under said first set of conditions, said biomass may be contacted with a solvent formulation under a second set of conditions.

Said second set of conditions may differ from said first set of conditions in at least one variable selected from:

(A) the physical state of the solvent formulation; and

(B) a chemical property of the solvent formulation.

As described in (A) above, the physical state of the solvent formulation may be varied during extraction of said biomass, for example in the receptacle. described The temperature or pressure of said solvent formulation may be varied. If the physical state of the solvent formulation is to be varied, it is preferred that the temperature of the solvent formulation is varied. For example, said first set of conditions may involve the solvent formulation being at a relatively low first temperature, as described. The temperature may be raised so that said solvent formulation is at a second temperature, greater than the first temperature. The temperature may be further raised so that said second set of conditions involve the solvent formulation being at a third temperature greater than the second temperature. Each of the temperatures of the solvent formulation may be within the range −10° C. to 60° C.

As described in (B) above, a chemical property of the solvent formulation may be varied during extraction of said mass of material in the receptacle. This may be achieved by including varying amounts of a selected modifier, as described herein, in said solvent formulation.

Preferably, in the method, when a second set of conditions is used, the physical state of the solvent formulation, especially the temperature thereof, is varied as described in (A) above.

A receptacle in which said biomass is preferably arranged in the method is preferably a column. The column preferably has a circular cross-section between its inlet and outlet. The column preferably has a substantially constant cross-sectional area and shape between its inlet and outlet. The column may have an inside diameter of at least 2 cm, preferably at least 4 cm, more preferably at least 5 cm. The diameter of the column may be less than 30 cm, preferably less than 15 cm. The length of the column between its inlet and outlet may be at least 40 cm, preferably at least 50 cm, more preferably at least 75 cm, especially at least 100 cm. The length of the column may be less than 500 cm, preferably less than 400 cm, more preferably less than 250 cm. The column may have a length: inside diameter ratio of greater than 1:1; and preferably less than 100:1. The length: diameter ratio may be at least 10, preferably at least 20. The column preferably has a circular cross-section over at least 50%, preferably 80% of its length. It preferably has a circular cross-section over substantially the entirety of its length. The length: inside diameter ratio may be less than 50:1. The column is preferably made out of metal, such as steel.

Said cannabinoid may be naturally-occurring in the biomass or may be a derivative of a cannabinoid which is naturally-occurring in the biomass. When said cannabinoid is a derivative, the method may include actively treating the biomass prior to step (i) of the method to derivative a naturally-occurring cannabinoid in the biomass.

In an embodiment (A), said biomass may not be actively treated prior to step (i) to derivatise a naturally-occurring cannabinoid in the biomass. For example, said biomass may not be actively treated to decarboxylate a naturally-occurring cannabinoid included in the biomass. A reference to “actively treated” or a cognate expression refers to a treatment which is operated or operable by a person using man-made equipment. It suitably excludes, for example, treatment and/or a reaction which occurs under ambient conditions, such as ambient temperature. Some cannabinoids may react, for example, decarboxylate to some extent under ambient conditions but, in the context, this is not regarded as an active treatment.

In embodiment (A), preferably, no component of said biomass is derivatised (e.g. oxidized or decarboxylated) in an active treatment prior to step (i) of the method.

In an embodiment (B), said biomass may be treated, for example actively treated, prior to step (i) to derivatise a naturally-occurring cannabinoid in the biomass. The biomass may be treated to decarboxylate one or more cannabinoid compounds included in the biomass. For example, the biomass may include tetrahydrocannabinolic acid (THCA) and/or cannabidiolic acid (CBDA). Such compounds may be decarboxylated, when within the biomass, to yield tetrahydrocannabinol (THC) and cannabidiol (CBD) respectively.

In embodiment (B), said biomass (suitably comminuted biomass) may be heated, for example to a temperature of greater than 80° C. or greater than 100° C., preferably in a substantially inert atmosphere (e.g. under a nitrogen blanket) for a period of time (e.g. in excess of 30 minutes or in excess of 1 hour) thereby to decarboxylate one or more major components in the biomass. After treatment of the biomass, it may be subjected to step (i) of the method as described.

Embodiment (A) may be preferred over embodiment (B).

Said biomass, prior to any decarboxylation, for example in an active treatment as described, may include THCA and/or CBDA. Preferably, in said biomass prior to any decarboxylation, for example in an active treatment as described, the sum of the wt % of THCA and CBDA is at least 0.5 wt %, suitably at least 3 wt %, preferably at least 10 wt %. Said sum may be less than 20 wt % or less than 15 wt %.

Said biomass may include, prior to any decarboxylation as described, at least 0.1 wt % of THCA and at least 0.1 wt % of CBDA. In said biomass, at least one of THCA or CBDA may be present at a level of at least 5 wt %, preferably at least 10 wt %, or at least 15 wt %.

After decarboxylation, the sum of the wt % of THC and CBD in the biomass may be at least 0.5 wt %, suitably at least 3 wt %, preferably at least 10 wt %. Said sum may be less than 20 wt % or less than 15 wt %.

After decarboxylation, said biomass may include at least 0.1 wt % of THC and at least 0.1 wt % of CBD. In said biomass after decarboxylation, at least one of THC or CBD may be present at a level of at least 5 wt %, preferably at least 10 wt %, or at least 15 wt %.

Said cannabinoid extracted in the method may include one or more cannabinoids selected from THC, THCA, CBD and CBDA.

Said biomass may be derived from a cannabis plant. Different strains of such plants may include different levels of THC and CBD. For example, selected strains may include the following:
High THC content, for example 3 wt %-22 wt % (or higher) and low CBD content;
Low THC, for example <0.5 wt %, and high CBD contents, for example and 3 wt %-12 wt % or higher;
High content of both THC and CBD;
Low THC, for example <0.3 wt %, and low CBD, for example 0.3 wt %-1 wt %.

Said biomass may be selected from Cannabis satira, Cannabis indica and Cannabis rudiralis and/or strains derived therefrom.

Said biomass treated in step (i) may have a water content of less than 10 wt % or less than 5 wt %.

The material extracted from the biomass and charged to the solvent is preferably a compound which occurs naturally in the biomass or is a derivative of a compound which occurs naturally in the biomass.

In the method, after step (ii), the charged solvent formulation may be treated so solvent formulation is separated from the extract, suitably to isolate the extract. Separation may simply involve evaporation of the solvent formulation.

The method may include preparing separate extracts from said biomass, for example, using said first set of conditions and a second set of conditions as described. The separate extracts may be isolated separately (thereby to provide extracts which may have different compositions, for example different amounts of THC, THCA, CBD and/or CBDA) or the separate extracts may be combined to define a single extract.

In an embodiment (C), a said extract (e.g. containing at least one cannabinoid, suitably selected from THC, THCA, CBD and CBDA) may be treated, for example, with a view to increasing the amount of one or more desired cannabinoids in a product of the treatment. Such treatment may comprise dissolution of an extract in a solvent (e.g. an organic solvent, such as ethanol) and/or subjecting the extract to a temperature less than ambient temperature (e.g. less than 0° C., less than −10° C. or less than −15° C.), for a period of time, for example at least 1 hour, at least 10 hours or at least 20 hours. The intention of the treatment is to cause precipitation of high molecular weight components (e.g. waxes) from the extract, thereby to increase the concentration of desired cannabinoids in the extract. After the treatment, the extract may be treated, for example filtered (e.g. to capture the high molecular weight components) and the filtrate collected thereby to define a purified extract, relatively rich in desired cannabinoids.

Whilst it is conventional to include treatment as described in embodiment (C) (which is often referred to as “winterization”), it is unexpectedly found, in accordance with preferred embodiments of the invention, that the method of the first aspect may be sufficiently selective so that significant quantities of high molecular weight components such as waxes, are not extracted in the method, meaning that, advantageously, the method described in embodiment (C) is not required. Thus, in an embodiment (D), the method preferably does not include a treatment of the extract as described in embodiment (C). In embodiment (D), the method preferably does not include one or more of the following:

(a) said extract (e.g. containing at least one cannabinoid, suitably selected from THC, THCA, CBD and CBDA) being treated, for example, with a view to increasing the amount of one or more desired cannabinoids in a product of the treatment;

(b) dissolution of an extract in a solvent (e.g. an organic solvent, such as ethanol);

(c) subjecting the extract to a temperature less than ambient temperature (e.g. less than 0° C., less than −10° C. or less than −15° C.), for a period of time, for example at least 1 hour, at least 10 hours or at least 20 hours;

(d) any other treatment intended to cause precipitation of high molecular weight components (e.g. waxes) from the extract;

(e) filtration (e.g. to capture the high molecular weight components).

Embodiment (D) preferably does not include at least two, at least three, at least four or any of steps (a) to (f) described. Embodiment (D) preferably does not include any treatment intended to cause or which does cause precipitation of high molecular weight components (e.g. waxes) from the extract.

Thus, in embodiment (D), the total weight of waxes in the charged solvent and/or the extract (e.g. containing at least one cannabinoid) derived therefrom is less than the total weight of waxes in the biomass after the biomass has been treated in the method. Waxes may be any lipophilic, organic compounds with a melting point of greater than 40° C. They are suitably substantially insoluble in water. The wax ratio, defined as the total weight of waxes in the biomass after treatment in the method divided by the total weight of waxes in the charged solvent, for example after step (ii) (and for the avoidance of doubt without any low temperature, for example winterization treatment as described in embodiment (C)) is suitably at least 5, preferably at least 10, more preferably at least 20, especially at least 40. Thus, the extract may include a very small amount of wax at most and as a result does not need to be subjected to a winterization treatment.

The extract, suitably the product of embodiment (D), may include a low level of waxes at most—e.g. less than 0.5 wt %, less than 0.25 wt %, less than 0.1 wt % or less than 0.05 wt %.

In the extract, the cannabinoid ratio, defined as the total weight of non-wax based cannabinoids divided by the total weight of waxes in the extract may be at least 5, preferably at least 10, more preferably at least 50, especially at least 100.

The extract, for example the product of embodiment (D), is preferably a mobile oil at 25° C.

When embodiment (A) is followed in the method, in preference to embodiment (B), the method may include an embodiment (E), wherein the extract is treated to derivatise a cannabinoid which was naturally-occurring in the biomass and is present in the extract. In this case, the extract may be treated to derivatise such a naturally-occurring cannabinoid in the extract. The extract may be treated to decarboxylate one or more cannabinoid compounds included in the extract. For example, the extract may include tetrahydrocannabinolic acid (THCA) and/or cannabidiolic acid (CBDA). Such compounds may be decarboxylated, when within the extract, to yield tetrahydrocannabinol (THC) and cannabidiol (CBD) respectively. In such treatment, said extract may be heated, for example to a temperature of greater than 80° C. or greater than 100° C., preferably in a substantially inert atmosphere (e.g. under a nitrogen blanket) for a period of time (e.g. in excess of 30 minutes or in excess of 1 hour) thereby to decarboxylate one or more major components in the extract. Preferably, in said extract before decarboxylation, the sum of the wt % of THCA and CBDA is at least 0.5 wt %, suitably at least 3 wt %, preferably at least 10 wt %. Said sum may be less than 20 wt % or less than 15 wt %.

After decarboxylation, said biomass may include at least 0.1 wt % of THC and at least 0.1 wt % of CBD. In said extract, at least one of THC or CBD may be present at a level of at least 5 wt %, preferably at least 10 wt %, or at least 15 wt %.

In a second aspect, the invention extends to an extract from a biomass as described per se. The extract may be as described in embodiment (D). The extract may include a low level of waxes at most—e.g. less than 0.5 wt %, less than 0.25 wt %, less than 0.1 wt % or less than 0.05 wt % of waxes. In the extract, the cannabinoid ratio, defined as the total weight of non-wax based cannabinoids divided by the total weight of waxes in the extract may be at least 5, preferably at least 10, more preferably at least 50, especially at least 100. The extract is preferably a mobile oil at 25° C.

Although the majority of said solvent formulation used in the method of the first aspect is separated from the extract, it is possible some of said solvent formulation may remain, for example as a contaminant, in the extract. Thus, said extract may include at least 0.0001 wt %,or at least 0.0010 wt %. of said solvent formulation, for example comprising a said a C₁₋₄ fluorinated hydrocarbon or a C₁₋₄ hydrofluorocarbon ether, Said extract may include less than 0.1000 wt %%, or less than 0.01 wt %, of a said solvent formulation.

Said extract may include at least 0.0001 wt % or at least 0.0010 wt %, of HFC134a. Said extract may include less than 0.1000 wt % or less than 0.01 wt %, of HFC134a.

In a third aspect, the invention extends to a formulation which comprises a product of the method of the first aspect and/or an extract of the second aspect.

The formulation may be for the following:
As a medically prescribed botanical drug substance;
As an OTC health related supplement. This may be for use as: a pain killer, a sleep aid, an antidepressant and an anti-anxiety remedy, anorexia treatment, a muscle relaxant, a spasticity aid, an anti-emetic, an appetite enhancer, an aid in some neurological disorders such as involuntary movements and vocalizations, a suppressant for involuntary tics and other Tourette symptoms;
Food additive, e.g. flavour enhancer;
Cosmetic and aroma ingredient;
Recreational uses.

In a fourth aspect, there is provided a method of making a formulation, for example according to the third aspect, the method comprising:
(a) selecting a product of the method of the first aspect and/or an extract of the second aspect;
(b) contacting material selected in step (a) with one or more other components of the formulation so as to incorporate a predetermined concentration of cannabinoids in the formulation;
(c) producing a mixture of said selected material and one or more other components.

The invention extends to the use of an extract of the second aspect, formulation of the third aspect or product of the method of the fourth aspect as a BDS, suitably for use in a medical treatment.

Any feature or any aspect of any invention or embodiment described herein may be combined with any feature of any aspect of any other invention or embodiment described herein mutatis mutandis.

Specific embodiments of the invention will now be described, by way of example, with reference to the accompanying diagrammatic drawings, in which:

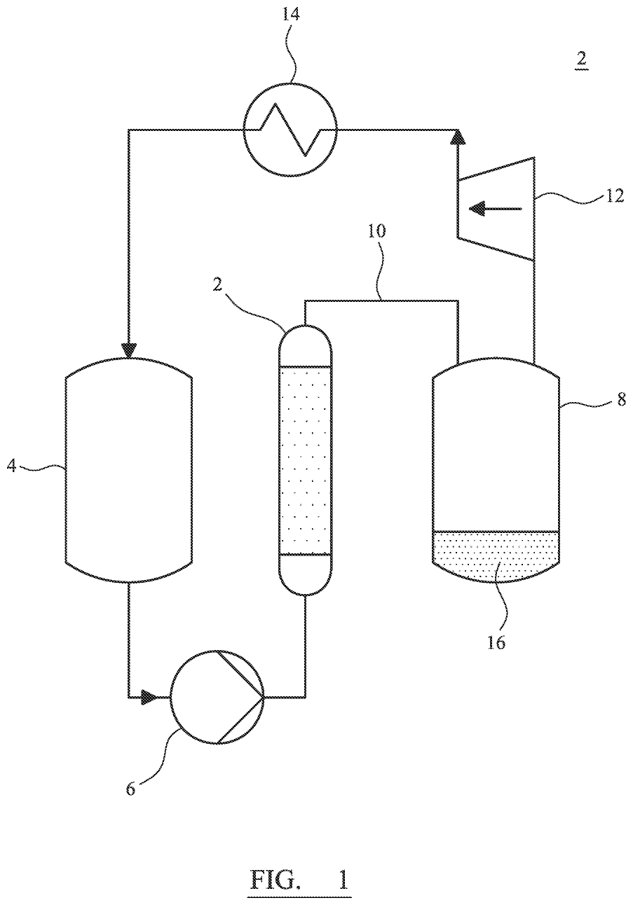

FIG. 1 is a schematic representation of apparatus for carrying out extraction of a cannabinoid-containing biomass;

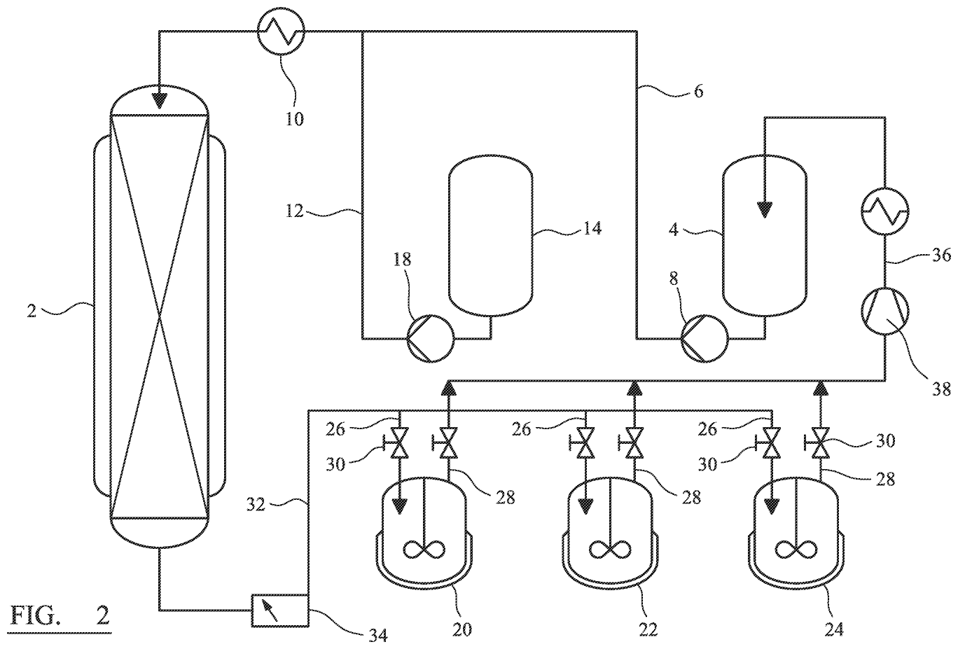

FIG. 2 is an alternative apparatus for carrying out extraction of a cannabinoid-containing biomass;

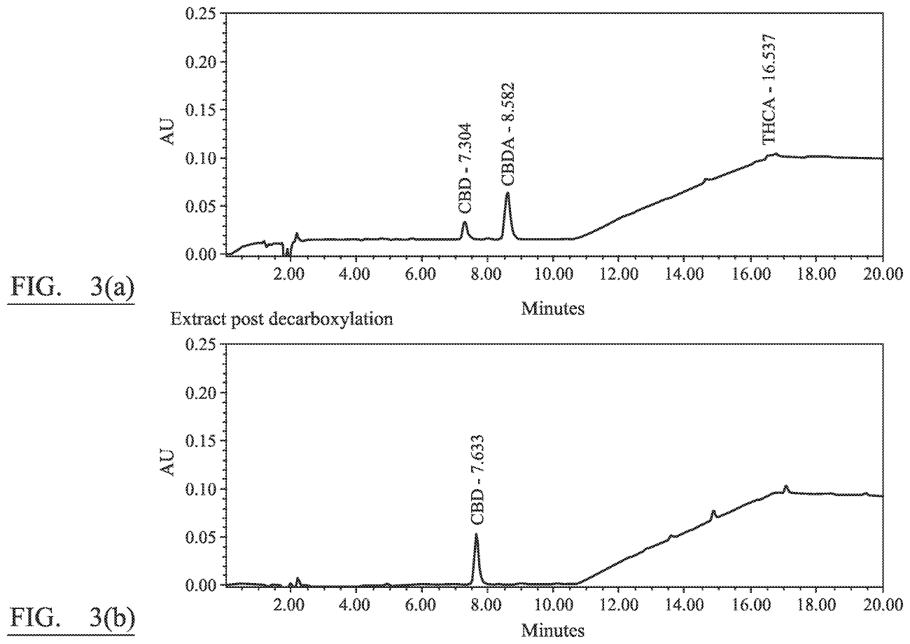

FIGS. 3(a) and 3(b) are chromatograms for an extract (Fraction No. 2) pre- and post-decarboxylation; and

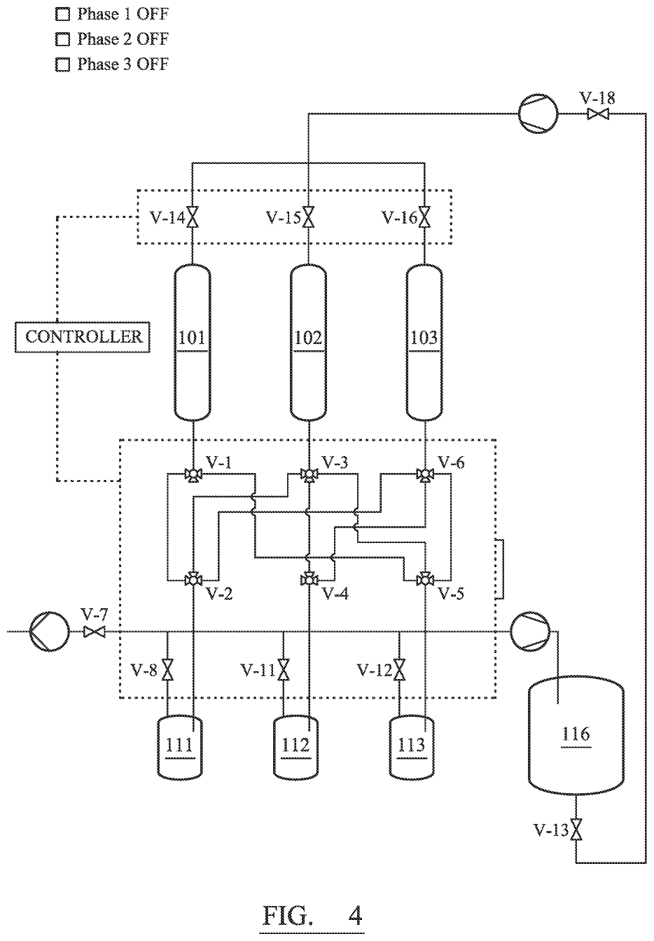

FIG. 4 is an alternative apparatus for use in a continuous extraction.

The following is referred to hereinafter:

R134—refers to 1,1,1,2-tetrafluoroethane.

Referring to FIG. 1, apparatus 2 for carrying out extraction of a biomass containing bio-cannabinoids comprises a jacketed stainless steel extraction column 2 having an internal diameter of 4.4 cm and a height of 1 m. Upstream of column 2 is a solvent storage vessel 4 with a liquid metering pump 6 being arranged between vessel 4 and column 2 for circulating liquid within the apparatus in which biomass to be extracted is tightly packed.

Downstream of column 2 is a collection/evaporation vessel 8 which communicates with the top of column 2 via pipe 10.

Downstream of vessel 8 is an oil free gas compressor 12 and a monitoring device (not shown) to monitor and analyse fluid flowing downstream of vessel 8. Downstream of the compressor and monitoring device 12 is an in-line heat-exchanger 14 which is arranged to re-liquefy fluid prior to return to vessel 4.

The apparatus described was used in examples which follow.

### Example 1—Decarboxylation of Cannabinoid-Containing Biomass

500 g of previously dried cannabis biomass was ground to a powder of average particle size of ≤1 mm and decarboxylated by placing in a sealed oven, and under a nitrogen blanket at 120° C. to 140° C. for 1-2 hours. In the process described, tetrahydrocannabinolic acid (THCA) which is a major component of the biomass is decarboxylated to produce tetrahydrocannabinol (THC) which is an active ingredient.

The biomass had a post-decarboxylation THC content of 5.9 wt %, measured by reverse-phase HPLC as described in Example 2, and a water content of 3 to 5 wt %.

### Example 2—Reverse Phase HPLC

The biomass and extracts were analysed using HPC as follows:

### Equipment Used

Waters 2695 separations module

### Detector: Waters PDA 996

Column: Phenomenex Kinetex C18 5 μm 250 mm×4.6 mm

### Mobile Phase Used

Mobile Phase A: Deionised water+0.1% formic acid
Mobile phase B: HPLC grade Methanol+0.1% formic acid
Gradient flow rate 1.0 ml/min:

### 0-10 min:20% A; 80% B

10-25 min:20% A; 80% B linear increase to 5% A; 95% B
25-26 min: back to 20% A; 80% B

### 26-28 min:20% A; 80% B

Injection volume: 10 ul
PDA scans from 200-400 nm—detection of cannabinoids at 220 nm

### Sample Preparation

### Plant Biomass:

200 mg is weighed in a centrifuge tube and 10.0 ml of methanol:ethyl acetate (9:1) is added.

### Cannabis Extract:

50 mg is weighed in a centrifuge tube, 10.0 ml of methanol:ethyl acetate (9:1) is added.

The tubes are vortexed for 15 seconds, then they are put in an ultrasonic bath for 15 minutes while vortexing every 5 minutes. The tubes are then centrifuged for 10 minutes at 5000 rpm. The supernatant is passed through a 0.45 um PTFE filter membrane and diluted to the right amount to fit in the calibration range.

### Example 3—Treatment of Biomass

The decarboxylated biomass from Example 1 was packed tightly in the extraction column 2 and the column was sealed. It was then cooled to −10° C. by placing in a freezer overnight. The equipment was then reassembled, evacuated and storage vessel 4 was charged with HFC134a (approx. 4-6 Kg). Chilled ethylene glycol was circulated through the jackets of the extraction column and the storage vessel, until the temperature of the HFC 134a in the storage vessel reached −5° C. The chilled HFC134a was then percolated through the biomass in column 2 at a flow rate of 5 litres/hour, with the flow being directed out of the column 2 and into vessel 8. The HFC134a was continuously evaporated from the vessel 8 using a gas compressor 12 in FIG. 1 and recycled through a condenser 14 in FIG. 1 back into the vessel 4.

The elution was carried out for two hours. At the end of the process, product 16, present in vessel 8, was harvested by dissolving it in a minimum volume of ethanol to produce “Extract Part 1” which was removed from vessel 16.

Elution was then continued except the temperature of the HFC 134a was increased to to 30° C. After a period of 4 hours, product 16 present in vessel 8 was harvested by dissolution in a minimum volume of ethanol to produce “Extract Part 2” which was removed from vessel 16.

### Example 4—Separation of Waxes and Isolation of Cannabinoids

The ethanolic solutions referred to as Extract Part 1 and Extract Part 2 were placed in a freezer at −20° C. to cause precipitation of non-target phytowaxes. The fluids were then filtered through a celite bed and subsequently the ethanol was removed by vacuum distillation to yield products high in desired cannabinoids, referred to as Isolated Extract A1 and Isolated Extract A2.

### Example 5—Treatment of Biomass Using a Solvent Formulation B

A decarboxylated biomass prepared as described in Example 1 was treated as described in Example 3 except that, instead of HFC 134c described in Example 3 being used as the sole solvent, a Solvent Formulation B was used which comprised HFC 134a and 5% wt/wt dimethylether as a co-solvent. The use of the co-solvent modifies the solvating properties of the HFC 134a. The process of Example 4 was used to yield desired cannabinoids, referred to as Isolated Extract B1 and Isolated Extract B2.

### Example 6—Treatment of Biomass Using a Solvent Formulation C

A decarboxylated biomass prepared as described in Example 1 was treated as described in Example 3 except that, instead of HFC 134c described in Example 2 being used as the sole solvent, a Solvent Formulation B was used which comprised HFC 134a and 5 wt % n-butane as a co-solvent. The use of the co-solvent modifies the solvating properties of the HFC 134a. The process of Example 4 was used to yield desired cannabinoids, referred to as Isolated Extract C1 and Isolated Extract C2.

### Example 7—Analysis of Isolated Extracts and Results

Isolated Extracts A1 and A2 were weighed and analysed for THC content (in g) and purity (%) by HPLC analysis, as described in Example 2.

Based on the biomass having a nominal post decarboxylation THC content of 5.9 wt %, a water content of 3 to 5 wt % and noting that 500 g used in Example 1 contains 29.5 g of THCA, extract purities were calculated as follows:

Weight of THC (g) in extract+Weight extracts (g)×100

Yields (wt %) were calculated as:

Weight (g) THC (g) in extract+29.5 (g)×100

Isolated Extracts B1 and B2 from Example 5 and Isolated Extracts C1 and C2 from Example 6 were analysed as described for Extracts A1 and A2.

Results are presented in Table 1.

TABLE 1

| Example | Extract | Weight | |
|---|---|---|---|
| No. | Reference | (g) | Observations |
| 4 | Isolated | 4.1 | Almost colourless solid. Some |
| | Extract A1 | | free water but no emulsion- |
| | | | very pure appearance |
| | Isolated | 26.3 | Yellow/honey colour, |
| | Extract A2 | | gummy oil |
| | | | Free water and light emulsion |
| 5 | Isolated | 6.7 | Light honey colour |
| | Extract B1 | | resinate/viscous oil. |
| | | | Free water light emulsion |
| | Isolated | 27.2 | Dark honey colour gummy oil, |
| | Extract B2 | | some free water, |
| | | | significant emulsion |
| 6 | Isolated | 7.1 | Honey colour resinate/ |
| | Extract C1 | | viscous oil. Light emulsion |
| | Isolated | 28.1 | Dark amber colour, gummy oil |
| | Extract C2 | | Free water and |
| | | | heavy emulsion present |

The purity of the extracts was analysed and results are provided in the table below.

TABLE 2

| | | Blended | Fraction | Purity % | THC |
|---|---|---|---|---|---|
| | | Ext. Wt. g | Wt. g | THC w/w | Yield % |
| 1 | Example 4 | 30.4 | 4.1 | Part1-95.3 | Calculated |
| | | | 26.3 | Part2-84.0 | 87.3 |
| 2 | Example 5 | 33.9 | 26.2 | 77.3 | 88.8 |
| 3 | Example 6 | 35.2 | 27.7 | 78.7 | 93.9 |

Note:
the blended Ext. Wt g for Example 4 is the sum of the two parts, 4.1 + 26.3 g.

### Example 8—Treatment of Biomass Rich in Cannabidiolic Acid (CBDA)

250 g of dried plant material with CBDA content of 3.9% and water content of 3-5% was decarboxylated as described in Example 1, milled to powder particle size of ≤1 mm and packed tightly into a stainless steel extraction column having dimensions of 3.5 cm internal diameter and 1 m length and then placed in a freezer overnight. The experiment was carried out generally using the apparatus described in FIG. 1 and as described in Examples 3 and 4. The extracts were collected in two fractions; part 1 after 1 hour at −6° C. and HFC134a flow rate of 3 Kg/Hr and part 2 collected after a further 6 hours at 25° C. In each case, the product was harvested from the evaporation vessel by dissolving in a minimum volume of ethanol. Both ethanolic fractions were treated as in Example 4.

Extracts were weighed and analysed to determine the CBD concentration using literature standard HPLC method. Results are presented in the table below, based on a theoretical amount of CBDA in the starting material of 9.75 g.

TABLE 3

| | | Weight | Weight | Purity | Yield | |
|---|---|---|---|---|---|---|
| Solvent | Ext. | g | CBD g | % | % | Appearance |
| HFC | Part1 | 2.63 | 2.60 | 98.9 | 26.67 | White sticky |
| 134a | | | | | | solid |
| | Part2 | 9.70 | 6.40 | 65.98 | 65.65 | Amber oil |
| | Total | | | | 92.3 | |

As an alternative to use of the apparatus of FIG. 1, the apparatus described in FIG. 2 may be used.

Referring to FIG. 2, apparatus for carrying out fractional extraction using a liquefied gas as extraction medium comprises an extraction column 102 in which material to be extracted is tightly packed. The column may be jacketed and include heating/cooling means for temperature control. Upstream of the column 102 is a hold vessel 104 for containing the liquefied gas, for example HFC 134a. The vessel 104 is connected downstream, by pipework 106, to the upper end of the column 102 for transferring the extraction medium into the column 102. A liquid metering pump 108 is provided in pipework 106 for controlling the flow of extraction solvent to the column. Immediately upstream of the column, the pipework includes an in-line heat exchanger 110 arranged to heat (or cool) liquid prior to its passage into the column. Between the pump 108 and heat-exchanger 110, there is a modifier solvent supply pipe 112 which is arranged to deliver a modifier solvent from a storage vessel 114 into the pipework 106 so that it mixes with liquefied gas from vessel 104. A liquid metering pump 116 in supply pipe 112 controls the flow of liquid within the pipe 112.

Downstream of the vessel 102 are shown three collection/evaporation vessels 120, 122, 124 although more such vessels would generally be provided for collecting more than three different aliquots. Each of the vessels 120, 122, 124 includes an inlet pipe 126 and an outlet pipe 128 each having associated control valves 130. The vessels 120, 122, 124 are arranged to communicate with column 102 via pipeline 132 which is connected to the bottom of the column. A monitoring device 134 is arranged to monitor and/or analyse fluid flowing in pipeline 132. Downstream of pipeline 132 is a pipeline 136 which communicates with vessel 104 and includes an associated gas compressor 138 for liquefying gas prior to its passage back into the vessel 104.

The apparatus further includes any necessary in-line filters, one-way valves, flow control valves, pressure regulators and pressure release valves and instrumentation for reading temperature, pressure and pH to allow appropriate process control and safe operation of the apparatus.

In use, a vacuum pump (not shown) is operated to remove air from the apparatus after the material to be extracted has been packed into the column 102. Liquefied gas is then charged to the vessel 104 and co-solvent, if this is used, is charged into vessel 114. With any heating/cooling means of the apparatus appropriately set, liquid is passed from vessel 104 to the column 102. The liquid slowly percolates through the material in the column and extracts compounds from the material as it does so. Initially, the most soluble compounds included in the biomass are extracted preferentially and these are entrained with liquid as it passes from the column into pipeline 132. The liquid (and entrained extract) is then directed into vessel 120 by opening the appropriate valve. After a period of time which may be determined in dependent upon an output from monitoring device 134, subsequent liquid passing out of the column is directed into vessel 122. Subsequently, it is directed into vessel 124 and later to other vessels (if provided). Thus separate aliquots are collected in vessels 120, 122, 124 and the constitution of the extracts therein should differ, with compounds or compositions which are most soluble in liquid passing through the column being more concentrated in the vessels which initially are used for collection and less soluble compounds or compositions being more concentrated in collection vessels used later in the process.

The constitution of extracts may also be affected by delivering a co-solvent from vessel 114 into pipeline 108 and mixing the co-solvent with liquid from vessel 104. The combined extraction solvent may then be adapted to extract preferentially certain compounds or compositions. The co-solvent may be delivered as described herein for manipulating the extraction of the biomass. Additionally and/or alternatively, the heat-exchanger 10 may be used to adjust the temperature of the extraction solvent thereby to control the nature of compounds or compositions preferentially extracted. Also, the temperature of the column itself (and thereby the biomass therein) may be adjusted as another means of affecting the nature of extracts.

After the extraction of the biomass has been completed (or prior to completion whilst extraction in the column 102 is ongoing), the control valve to outlet pipe 128 of vessel 120 may be opened and compressor 138 operated to remove liquefied solvent from the vessel 120 and return it to vessel 104, leaving the compound(s)/composition(s) in vessel 20. This process may be repeated to isolate the different extracts in the respective vessels 120, 122, 124.

The aforementioned examples involve treatment of a cannabinoid-containing biomass which has been decarboxylated (as is conventional in industry) as described in Example 1. However, in some cases, it is found that such decarboxylation can result in charring of the plant material, a darkening in its colour and production of acrid smoke. This may also result in loss of cannabinoid content and production of lower purity extract. Example 8 which follows describes treatment of biomass prior to any decarboxylation.

### Examples 8 and 9—Comparison of Treatments of Non-Decarboxylated and Decarboxylated Cannabis Biomasses

In both examples, respective samples of the same botanical material having a CBDA content of 7.5 wt % were treated.

In Example 8, the botanical material was treated as described, without any decarboxylation. In Example 9, the botanical material was decarboxylated prior to treatment as described in Example 1. The biomasses of Examples 8 and 9 were treated generally as described in Example 3, with, in each case, respective fractions being collected at different temperatures of HFC 134a as described in Table 4 below.

TABLE 4

| | Temperature of HFC 134a at | |
|---|---|---|
| Fraction No. | time of collection of fraction | |
| 1 | −10°⁻ | C. |
| 2 | 0° | C. |
| 3 | 25° | C. |

Fractions were analysed for cannabinoid content using HPLC as described in Example 2. Results are provided in Table 5.

TABLE 5

| | Example No. 8 | | Example No. 9 | |
|---|---|---|---|---|
| | Weight (g) | CBDA (wt %) | Weight (g) | CBD (wt %) |
| Biomass | 166.8 | 7.5 | 159.7 | 5.2 |
| Spent biomass | 143 | 0.8 | 146.5 | 0.5 |
| Fraction No. 1 | 0.62 | 66.3 | 1.32 | 57.9 |
| Fraction No. 2 | 12.9 | 59.9 | 8.55 | 53.3 |
| Fraction No. 3 | 4.32 | 64.3 | 1.01 | 50.7 |
| Wash | | | 370 | 0.4 |
| Total extracts | 17.84 | | 10.88 | |
| Total recovery | | 92.0 | | 89.0 |

It was noted that the fractions for Example 8 were uniformly lighter in colour and less viscous compared to those for Example 9. In addition, better recovery was achieved for Example 8.

Note that no “wash” is quoted for Example 8 because the product of Example 8 is a mobile oil whereas Example 9 is a sticky gum and, accordingly, a wash is needed to harvest all extract.

### Example 10—Decarboxylation of the Material from Example 8

Decarboxylation of Example 8, Fraction No. 2 was carried out by exposing the sample at a temperature of 180° C. for 10 minutes, as described in Example 1. HPLC analysis showed that 100% decarboxylation was achieved in total theoretical yield, determined as follows:

CBD content (by HPLC)=Original CBDA content

314.5 (molecular weight) 358.5 (molecular weight)

Reference is made to FIGS. 3(a) and 3(b). The CBDA peak is lost on decarboxylation as decarboxylation yields CBD, the peak for which is increased as per FIG. 3(b). FIGS. 3(a) and (b) demonstrate that total decarboxylation is readily achieved when the process of Example 4 (referred to as “winterization”) is carried out.

Examples 3 to 6 above include a conventional “winterization” step, as described in Example 4, to precipitate non-target phytowaxes and/or heavy fatty acids. Example 11 describes an alternative process.

### Example 11—Assessment of Winterization of CBD-Rich Extracts

CBD-rich primary extract produced in a method analogous to that described in Example 3 was dissolved in ethanol in a series of concentrations and the resulting solutions stored in a freezer at −20° C. for 24 hours as per Example 4. The solutions were then clarified by filtration and ethanol was removed by evaporation under a vacuum. The starting materials and products were analysed using reverse phase HPLC.

Table 6 shows the amount of primary extract used in each sample 1 to 8, the amount of ethanol used to dissolve the extract, the CBD content in each of the products and the loss in CBD content.

TABLE 6

| | Amount of | Ethanol | CBD content | Loss in CBD |
|---|---|---|---|---|
| Sample | extract (g) | ml | mg/g | Content % |
| Primary extract | | | 659 | |
| 1 | 2.5 | 3.5 | 635 | 4 |
| 2 | 2.5 | 5.25 | 615 | 7 |
| 3 | 2.5 | 7.0 | 598 | 9 |
| 4 | 2.5 | 10.5 | 622 | 6 |
| 5 | 2.5 | 34.0 | 603 | 8 |
| 6 | 2.5 | 28.0 | 587 | 11 |
| 7 | 1.15 | 28.0 | 440 | 19 |
| 8 | 1.7 | 26.0 | 455 | 16 |

The following observations were made:
Material precipitated on freezing was observed to be light and fluffy in appearance.
Only minimal precipitation was noted when small volumes of ethanol were used (Samples 1, 2 and 3)
Heavier precipitation clearly noted when higher volumes of ethanol were used (Samples 6, 7, and 8).
No change in viscosity or colour was observed in any of the treated samples when compared to the untreated primary extract.
All trials resulted in appreciable loss of CBD.

It is clear from the above observations that no waxes or heavy fatty acids were precipitated as expected and that the products that were precipitated during freezing were CBD. Therefore, in preferred embodiments, winterization (e.g. as described in Example 4) may not be necessary and may be disadvantageous. Thus, the process described herein can be used to make high purity products in high yield without the need to isolate an intermediate which needs further treating in a winterization step.

### Example 12—Further Analysis of Extracts

A number of previously prepared extracts were submitted for external contract analysis by a validated third party in order to identify some of the spectrum of compounds present in the extracts and also to validate results reported in the foregoing examples. Table 7 provides results of an analysis of an extract prepared as described herein of decarboxylated Bedrocan hybrid biomass rich in cannabidiol.

TABLE 7

| | Amount determined in |
|---|---|
| Compound assessed | analysis by third party |
| in analysis | (wt %) |
| CBD | 85.139 |
| d8-THC | 1.273 |
| d9-THC | 3.544 |
| CBG | 0.200 |
| CBN | 0.913 |
| CBC | 3.394 |
| CBDV | 1.897 |
| THCV | 0.185 |
| Total cannabinoids | 96.5 |
| Total cannabinoid | 11.4 |
| content other than CBD | |

It was found, in general, that the analysis by the third party identified higher levels of desirable extracted compounds (eg >10 wt % higher) than identified by Applicant. Thus, Applicant believes the process described may be even more advantageous than implied by the results in prior examples herein.

In addition, the third party analysis identified a range of other compounds (identified in Table 7).

In a further analysis, another extract of decarboxylated Bedrocan hybrid biomass rich in cannabidiol was analysed for terpenes and it was determined that desirable terpene compounds were extracted, as detailed in Table 8. Such compounds may act synergistically with cannabinoids to produce an advantageous mixture which may be highly useful as a BDS.

TABLE 8
Amounts of terpenes present in
analysis by third party

| Terpene | Content % |
|---|---|
| Alpha-Pinene | 0.36 |
| Beta-Pinene | 0.21 |
| Delta-3-Carene | 0.12 |
| Eucalyptol | 0.46 |
| Beta-caryophyllene | 1.04 |
| Myrcene | 2.17 |
| limonene | 0.06 |
| linalool | 0.04 |
| terpineol | 0.04 |
| humulene | 0.32 |

Total terpenes present = 4.8 wt %.

As an alternative to the apparatus described above, a continuous extraction process may be undertaken, as described below with reference to FIG. 4.

The apparatus includes a series of between two to five extraction columns, shown in diagrams 101, 102 and 103, all being of the same size and each being fitted with a jacket that allows heating/cooling of each column independently. Each column has a working volume of between 0.5 L-100 L, preferably between 2-50 L and dimension of between 5 cm-50 cm diameter and between 80 cm-150 cm length. Each column incorporates an inlet and an outlet valve and is fitted with a sampling port at its outlet. Optionally, a second flow inlet valve may be incorporated to each column inlet to allow the introduction of a co-solvent (not shown).

There are optionally between 2-5 evaporation/extract collection vessels, shown in the diagram as 111, 112, and 113. The vessels are jacketed to allow heating/cooling independently of each other and are fitted with flow restriction nozzles at their inlets to control pressure drops. The size of each vessel is determined by the scale of operation and may nominally be between 2 L-50 L in volume.

A solvent recycling vessel shown in the diagram as 116 is fitted with a heating or cooling jacket and has a size determined by the scale of operation. It may optionally have a volume of between 5 L-200 L.

The equipment also incorporates two, or optionally up to four, oil free compressors, each with a gas flow capacity of between 50 Kg/Hr-1000 Kg/Hr dependant on the scale of operation. It also includes two or optionally up to four liquid feed pumps each with a capacity of between 50 Kg/Hr-1000 Kg/Hr. The primary elements of the equipment are in communication with each other via a programmable system of heat exchangers, flow control valves, flow measuring devices, thermometers, pressure gauges and pressure release valves to allow flexible and safe control of flows, operating pressures and temperatures that are appropriate in each element of the equipment at each stage of the process. All the elements of the apparatus are in communication with each other via a series of programmable valves (V−1 to V18) to allow a continuous extraction operation.

The following describes a production scale continuous extraction process comprising your columns (the fourth column has been omitted from FIG. 4) and three extract fractions collected in three collection vessels. For the purpose of clarity, flows through each column are shown to be from top to bottom (down-flow). It is however preferable in practice to carry out the extraction in an up-flow mode. Also, not all process valves and process controls and instrumentation are shown.

Each column is packed with previously dried and powdered cannabis biomass. The cannabis beds are packed down tightly using mechanical means and the columns reassembled. The apparatus is then sealed and air is removed from the entire apparatus with the aid of a vacuum pump. Vessel 116 is then isolated and charged with the extraction medium, a hydrofluorocarbon such as HFC134a. Cooling and heating fluids are pumped through the various jackets to attain the required temperature in each element of the equipment. Once required temperatures are attained the extraction is commenced as follows:

Step 1:

With valves V13, V18, V14, open and all other valves closed liquefied, extraction medium is charged from the recycling vessel 116 to column 101 using the liquid flow pump. When column 101 is full, valves V1, V2 (inlet to vessel 101) and valve V8 are opened and flow is maintained using the appropriate flow, temperature and pressure control measures to maintain a pre-determined steady state. Extract flow samples are collected and analysed at regular intervals. With the aid of the compressor, the extraction medium is continuously evaporated, re-liquefied and charged back into recycling vessel 116.

At an appropriate stage, for example when a first component has been eluted from column 101, the temperature/pressure conditions (or optionally the extraction fluid composition) is altered to enable the elution of a second component. Simultaneously, flow through valve V−2 is diverted into vessel 112, valves V−4 and V−7 are opened and second compressor is started.

Step 2: Whilst extract from column 101 is collecting in vessel 112, extraction of column 102 is commenced using a second liquid flow pump directing the eluate into vessel 111 (following the procedure described in step 1). Now fraction 2 eluating from column 101 is collecting in vessel 112 and column 102 is collecting fraction 1 in vessel 111. At an appropriate time, temperature/pressure conditions (or optionally extraction fluid compositions) are altered.

Step 3: Valves sequence is changed so that flow from column 101 is redirected to collect extract fraction 3 in vessel 113 and the flow from column 102 is redirected to collect fraction 2 in vessel 112 and column 103 extraction is commenced directing the flow from 102 into vessel 111. At an appropriate time, flow into column 101 is terminated and temperature/pressure conditions (or optionally extraction fluid compositions) for 102 and 103 are altered. Flow out of column 101 continues until its now spent biomass content is completely degassed.

Step 4: Flow from column 102 is directed to vessel 113 and from column 103 into vessel 2. Extraction of a fourth column (not shown) is commenced and, as previously, fraction one is collected in vessel 111. Simultaneously, column 101 is cleaned, repacked and made ready for extraction in step 5, and so on.

The process steps described are shown in a simplified form below

| | Step 1 | Step 2 | Step 3 | Step 4 | Step 5 |
|---|---|---|---|---|---|
| Vessel 111 | Col 101 | Col 102 | Col 103 | Column 4 | Col 101 |
| Vessel 112 | X | Col 100 | Col 102 | Col 103 | Column 4 |
| Vessel 113 | X | X | Col 101 | Col 102 | Col 103 |
| Column | X | X | X | Col 101 | Col 102 |
| Repacking | | | | | |

The process sequence is controlled by a programmable system of valves and flow, temperature and pressure control measures. The process may require minimal operator involvement.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1-41. (canceled)

42. A method of extracting at least one cannabinoid from a biomass, the method comprising the following steps:
(i) contacting the biomass with a solvent formulation which comprises a C₁₋₄ fluorinated hydrocarbon or a C₁₋₄ hydrofluorocarbon ether, thereby to charge the solvent formulation with an extract from the biomass; and
(ii) separating charged solvent formulation from the biomass.

43. The method according to claim 42, wherein, in the method, said biomass is arranged in a receptacle between an inlet and outlet of the receptacle and, in the method, solvent formulation passes into the receptacle via said inlet, through the biomass and out of the receptacle via said outlet, wherein the method involves selecting a biomass which includes components to be extracted and packing the biomass into free space in said receptacle so that said biomass extends over a length of at least 40 cm in said receptacle, wherein said receptacle is a column.

44. The method according to claim 43, wherein said biomass is packed into said receptacle at a density of at least 0.25 g/cm³, wherein said biomass is substantially immovable when in position and/or the biomass is substantially static during the flow of said solvent formulation therethrough.

45. The method according to claim 44, wherein said solvent formulation is passed through the biomass at a rate of at least 0.02 ml/minute per gram of said biomass; and said rate is less than 1 ml/minute per gram.

46. The method according to claim 43, wherein the flow rate of solvent formulation through the biomass is at least 0.5 BV/hour where “By” refers to the bed volume.

47. The method according to claim 42, wherein said biomass is arranged in a column which has an inside diameter of at least 5 cm and an inside diameter of less than 30 cm; wherein the length of the column between its inlet and outlet is at least 100 cm and is less than 500 cm, wherein the column has a length: inside diameter ratio of greater than 10:1 and less than 100:1.

48. The method according to claim 47, wherein said solvent formulation comprises a said C₁₋₄ fluorinated hydrocarbon which is non-chlorinated.

49. The method according to claim 44, wherein said solvent formulation comprises 1,1,1,2-tetrafluoroethane.

50. The method according to claim 49, wherein in said biomass prior to any decarboxylation, the sum of the wt % of THCA and CBDA is at least 10 wt %; and said sum is less than 20 wt %; and/or
wherein, in said biomass, at least one of THCA or CBDA is present at a level of at least 15 wt %; and
wherein said cannabinoid extracted in the method includes one or more cannabinoids selected from THC, THCA, CBD and CBDA.

51. The method according to claim 42, wherein said biomass is derived from a cannabis plant and/or said biomass, prior to any decarboxylation, includes THCA and/or CBDA.

52. A method according to claim 42, wherein the method does not include one or more of the following:
(a) subjecting an extract to a temperature less than 0° C. for a period of time of at least 1 hour;
(b) treating the extract and precipitating high molecular weight components comprising waxes from the extract;
(c) filtering the extract.

53. A method according to claim 42, wherein the total weight of waxes in the charged solvent and/or an extract derived therefrom is less than the total weight of waxes in the biomass after the biomass has been treated in the method and/or after step (ii).

54. A method according to claim 42, wherein the wax ratio, defined as the total weight of waxes in the biomass after treatment in the method and/or after step (ii) divided by the total weight of waxes in the charged solvent, for example after step (ii), is at least 40.

55. The method according to claim 42, wherein an extract produced after step (ii) after removal of said solvent formulation comprises less than 0.5 wt % total waxes.

56. The method according to claim 49, wherein an extract produced after step (ii) after removal of said solvent formulation includes less than 0.5 wt % or less than 0.05 wt % total waxes.

57. The method according to claim 47, wherein, in the extract and/or charged solvent formulation, the cannabinoid ratio, defined as the total weight of non-wax based cannabinoids divided by the total weight of waxes in the extract is at least 100.

58. The method according to claim 42, wherein an extract produced after step (ii) after removal of said solvent formulation is a mobile oil at 25° C.

59. The method according to claim 45, wherein said solvent formulation comprises 1,1,1,2-tetrafluoroethane and wherein the wax ratio, defined as the total weight of waxes in the biomass after treatment in the method and/or after step (ii) divided by the total weight of waxes in the charged solvent after step (ii), is at least 40.

60. An extract from a biomass, wherein said extract includes less than 0.05 wt % of waxes, a cannabinoid ratio, defined as the total weight of non-wax based cannabinoids divided by the total weight of waxes, of at least 100;
wherein said extract is a mobile oil at 25° C. and includes at least 0.0001 wt % of HFC134a.

61. A method of extracting at least one cannabinoid from a biomass, the method comprising the following steps:
(i) contacting the biomass with a solvent formulation which comprises 1,1,1,2-tetrafluoroethane thereby to charge the solvent formulation with an extract from the biomass; and
(ii) separating charged solvent formulation from the biomass;
wherein, in the method, said biomass is arranged in a receptacle between an inlet and outlet of the receptacle and, in the method, solvent formulation passes into the receptacle via said inlet, through the biomass and out of the receptacle via said outlet, wherein the method involves selecting a biomass which includes components to be extracted and packing the biomass into free space in said receptacle so that said biomass extends over a length of at least 40 cm in said receptacle, wherein said receptacle is a column;
wherein said biomass is packed into said receptacle at a density of at least 0.25 g/cm³, wherein said biomass is substantially immovable when in position and/or the biomass is substantially static during the flow of said solvent formulation therethrough;
wherein said solvent formulation is passed through the biomass at a rate of at least 0.02 ml/minute per gram of said biomass; and said rate is less than 1 ml/minute per gram;
wherein said biomass is arranged in a column which has an inside diameter of at least 5 cm and an inside diameter of less than 30 cm; wherein the length of the column between its inlet and outlet is at least 100 cm and is less than 500 cm, wherein the column has a length: inside diameter ratio of greater than 10:1 and less than 100:1;
wherein the wax ratio, defined as the total weight of waxes in the biomass after treatment in the method and/or after step (ii) divided by the total weight of waxes in the charged solvent after step (ii) is at least 40;
wherein an extract produced after step (ii) after removal of said solvent formulation includes less than 0.5 wt % total waxes.
